(19) United States
(12) Patent Application Publication
(10) Pub. No.: US 2024/0082797 A1
(43) Pub. Date: Mar. 14, 2024

(54) UREA WATER MANUFACTURING DEVICE AND THEREOF METHOD

(71) Applicant: Jaekwang Seo, Uiwang-si (KR)

(72) Inventors: Woo-Sik JEONG, Incheon (KR); Hyun Bong, Seoul (KR); Sung Wook Lee, Seoul (KR)

(21) Appl. No.: 18/389,295

(22) Filed: Nov. 14, 2023

(30) Foreign Application Priority Data
Feb. 27, 2019 (KR) 10-2019-0023345

(51) Int. Cl. B01F 35/221; B01F 21/10; B01F 27/808; B01F 31/80; B01F 31/85; B01F 35/13; B01F 35/213; B01F 35/22; C05C 9/00; C05G 5/23; C07C 273/14

(52) U.S. Cl. CPC B01F 35/2211; B01F 21/10; B01F 27/808; B01F 31/83; B01F 31/85; B01F 35/13; B01F 35/213; B01F 35/2202; B01F 35/2215; C05C 9/00; C05G 5/23; C07C 273/14; B01F 2101/2204

(57) ABSTRACT

An exemplary embodiment of the present invention provides a urea water manufacturing device which can reduce the time for producing urea water by forming a vibrating atmosphere using an ultrasonic wave generator when stirring urea and pure water supplied inside a stirring tank, and can produce urea water with high purity by real-time feedback control of specific gravity of urea water, and a method thereof. The urea water manufacturing device according to an exemplary embodiment of the present invention includes a pure water supply unit, a urea supply unit, a stirring unit, a specific gravity detection unit, a control unit, and a urea water discharge unit.

### CROSS-REFERENCE TO RELATED APPLICATION

This application is a continuation-in-part application of application Ser. No. 16/784,348, filed Feb. 7, 2020, which claims priority to and the benefit of Korean Patent Application No. 10-2019-0023345 filed in the Korean Intellectual Property Office on Feb. 27, 2019, the entire contents of which are incorporated herein by reference.

### BACKGROUND OF THE INVENTION

### (a) Field of the Invention

The present invention relates to a urea water manufacturing device and a method thereof, and an automatic urea water vending machine.

### (b) Description of the Related Art

Due to tightening regulations on nitrogen oxide emissions, the distribution of diesel vehicles with selective catalytic reduction (SCR, a urea water supply unit) is gradually expanding. Urea water is a chemical material prepared by mixing pure water with urea, which is a raw material of urea fertilizer, and can be commercially available in Korea only after it passes the test in accordance with Article 74, Paragraph 2 of the Clean Air Conservation Act of Korea. Urea is a polar material that is dissolved easily when heated, but urea has a drawback in that impurities such as biuret and triuret are also dissolved together therein. Urea water in which biuret and triuret are dissolved causes corrosion and blockage in SCR devices. To precipitate and purify these impurities, it is necessary to remove them using a filter after dissolving at a low temperature, although the process takes some time.

KR Patent No. 10-1650399 (Air Injection Type Apparatus for Urea Solution and Manufacturing Method Thereof) discloses this low temperature dissolving, but an additional device such as a compressor for low temperature dissolving is required. Therefore, to manufacture a urea water production device with a size of about a water purifier, it is required to develop a technology of low temperature dissolving that can increase the dissolution rate within a stirring tank without the need of a large-volume auxiliary device.

The above information disclosed in this Background section is only for enhancement of understanding of the background of the invention and therefore it may contain information that does not form the prior art that is already known in this country to a person of ordinary skill in the art.

### SUMMARY OF THE INVENTION

An exemplary embodiment of the present invention provides a urea water manufacturing device which can reduce the time for producing urea water by forming a vibrating atmosphere using an ultrasonic wave generator when stirring urea and pure water supplied inside a stirring tank, and can produce and sell urea water with high purity by real-time feedback control of specific gravity of the urea water, a method thereof, and an automatic urea water vending machine.

A urea water manufacturing device, which includes: a pure water supply unit, which includes a pure water supply line connected with a stirring tank and that supplies pure water into the stirring tank; a urea supply unit, which includes a urea supply line connected with the stirring tank and that supplies urea into the stirring tank; a stirring unit, in which the pure water and the urea supplied into the stirring tank are stirred in a predetermined stirring vibration atmosphere; a specific gravity detection unit, which detects specific gravity of urea water produced by mixing of pure water and urea being stirred inside the stirring tank and generates a corresponding specific gravity detection signal thereof; a control unit, which analyzes the specific gravity detection signal that is generated in and supplied from the specific gravity detection unit, compares the detected specific gravity of the urea water with a predetermined discharge range of specific gravity, and generates a corresponding feedback control signal thereof so that the detected specific gravity of the urea water is included in the predetermined discharge range of specific gravity, thereby controlling the specific gravity of urea water produced inside the stirring tank; and a urea water discharge unit, which includes a urea water discharge line connected with the stirring tank and that discharges the urea water produced inside the stirring tank to the outside of the stirring tank through the urea water discharge line.

The specific gravity of urea water discharge may be set in a range of 1.305 sg to 1.315 sg. In particular, when the detected specific gravity of the urea water is detected to exceed 1.315 sg, the control unit can perform a series of control actions of supplying a pure water supply feedback control signal to the pure water supply unit and thereby further supplying pure water into the stirring tank. In addition, when the detected specific gravity of the urea water is detected to be lower than 1.305 sg, the control unit can perform a series of control actions of supplying a urea supply feedback control signal to the urea supply unit and thereby further supplying urea into the stirring tank.

The stirring unit includes: a stirrer, which rotates as an external force is transmitted thereto and thereby stirs pure water and urea; a stirring operation unit, which generates rotational power of the stirrer; and an ultrasonic wave generator that generates vibration waves; and may include a stirring vibration unit, which forms a stirring vibration atmosphere for stirring of the pure water and the urea inside the stirring tank.

The pure water supply unit may include: a pure water filtration unit, which is provided on the inlet side of the pure water supply line to filter the pure water being supplied from the outside; a pure water supply control unit, which is provided in the pure water supply line to control the supply amount of pure water filtered through the pure water filtration unit; an injection unit, which is provided at the outlet side of the pure water supply line and injects pure water; and a temperature control unit, which is provided between the pure water filtration unit and the pure water supply control unit in the pure water supply line, and controls the temperature of pure water to a predetermined supply temperature.

The urea supply unit may include: a urea storage unit, which stores urea supplied to the stirring tank; a particle forming unit, which forms the urea stored in the urea storage unit in particles; and a urea supply control unit, which supplies an accurate amount of the particulated urea into the stirring unit. The device may further include a filtration unit, which is provided in the urea water discharge line and filters impurities being discharged through the urea water discharge line.

The device may further include a cleaning unit, which includes a cleaning line connected with the pure water supply line and the urea water discharge line, and that selectively removes the urea water that remains in the stirring tank or the urea water discharge line using the pure water supplied from the pure water supply line.

A urea water manufacturing method according to an exemplary embodiment of the present invention may include: supplying pure water into a stirring tank through a pure water supply line connected with the stirring tank; stirring the pure water and the urea, which are supplied into the stirring tank, in a predetermined stirring vibration atmosphere; detecting specific gravity of urea water produced by mixing of pure water and urea being stirred inside the stirring tank and generating a corresponding specific gravity detection signal thereof; comparing the specific gravity of the urea water that is detected in the step of detecting specific gravity with a predetermined discharge range of specific gravity, and generating a corresponding feedback control signal thereof so that the detected specific gravity of the urea water is included in the predetermined discharge range of specific gravity, thereby controlling the specific gravity of urea water produced inside the stirring tank; and discharging the urea water produced inside the stirring tank to the outside of the stirring tank through the urea water discharge line connected with the stirring tank.

In particular, the specific gravity of urea water discharge is set in a range of 1.305 sg to 1.315 sg, and when the specific gravity of the urea water detected in the step of controlling the specific gravity of urea water is detected to exceed 1.315 sg, the method may further include a step of further supplying pure water, in which pure water is further supplied into the stirring tank through the pure water supply line by generating a pure water supply feedback control signal; and in addition, when the detected specific gravity of the urea water in the step of controlling the specific gravity of urea water is detected to be lower than 1.305 sg, the method further may further include a step of further supplying urea, in which urea is further supplied into the stirring tank through the urea supply line by generating a urea supply feedback control signal.

The method may further include a step of cleaning, in which the urea water remaining in the stirring tank or the urea water discharge line are selectively removed through the cleaning line, being connected with the pure water supply line and the urea water discharge line, using pure water supplied from the pure water supply line.

An automatic vending machine of urea water according to an exemplary embodiment of the present invention includes: a pure water supply unit, which includes a pure water supply line connected with a stirring tank and that supplies pure water into the stirring tank; a urea supply unit, which includes a urea supply line connected with the stirring tank and that supplies urea into the stirring tank; a stirring unit, in which the pure water and the urea supplied into the stirring tank are stirred in a predetermined stirring vibration atmosphere; a specific gravity detection unit, which detects specific gravity of urea water produced by mixing of pure water and urea being stirred inside the stirring tank and generates a corresponding specific gravity detection signal thereof; a control unit, which analyzes the specific gravity detection signal that is generated in and supplied from the specific gravity detection unit, compares the detected specific gravity of the urea water with a predetermined discharge range of specific gravity, and generates a corresponding feedback control signal thereof so that the detected specific gravity of the urea water is included in the predetermined discharge range of specific gravity, thereby controlling the specific gravity of urea water produced inside the stirring tank; a case, which is provided in a middle portion thereof with a urea water discharge unit, which includes a urea water discharge line connected with the stirring tank and that discharges the urea water produced inside the stirring tank to the outside of the stirring tank through the urea water discharge line; a urea storage unit, which is provided in an upper portion inside the case and supplies urea to the urea water manufacturing device; and a vend product outlet, which is provided in a lower portion inside the case and extracts the urea water which is manufactured from the urea water manufacturing device and productized.

The automatic vending machine of urea water may further include: a cost processing unit, which is able to handle costs with money or credit card; a urea water display unit, which displays a sample of a predetermined urea water product; a selection unit, which is able to select the urea water product; and a display unit, which displays information related to manufacture of the urea water product.

The automatic vending machine of urea water, which includes a urea water injection device for injecting urea water, which is manufactured from the urea water manufacturing device and productized, into a device, wherein the urea water injection device includes a urea water injection gun and an injection hose, which connects the case with the urea water injection gun.

The automatic vending machine of urea water connects the urea water, which is manufactured from the urea water manufacturing device and productized, into a selective catalytic reduction (SCR) device or selective non-catalytic reduction (SNCR) device for incinerators, through a supply pipe.

The automatic vending machine of urea water has effects of reducing the time for producing urea water by forming a vibrating atmosphere using an ultrasonic wave generator when stirring particulated urea and pure water supplied inside a stirring tank, and producing urea water with high purity by real-time feedback control of specific gravity of urea water.

The automatic vending machine of urea water has effects of realizing down-sizing of the urea water manufacturing device by reducing unnecessary additional facilities and processes when manufacturing urea water, and manufacturing high-quality urea directly wherever and whenever needed without any restriction on place and time via real-time feedback control of specific gravity of urea water.

The automatic vending machine of urea water has the effect of manufacturing urea water with high purity by easily cleaning the stirring tank and the urea water discharge line when manufacturing urea water.

### BRIEF DESCRIPTION OF THE DRAWINGS

FIG. 1 is a drawing illustrating a urea water manufacturing device according to an exemplary embodiment of the present invention.

FIG. 2 is a block diagram illustrating the control relationship of the urea water manufacturing device according to the present invention.

FIG. 3 is a block diagram illustrating the process of discharging urea produced by the supply of urea and pure water in the process of manufacturing urea according to an exemplary embodiment of the present invention.

FIG. 4 is a schematic diagram illustrating the process of cleaning a stirring tank using a cleaning unit and an ultrasonic wave generator according to an exemplary embodiment of the present invention.

FIG. 5 is a schematic diagram illustrating the process of cleaning a urea water discharge line using a cleaning unit according to an exemplary embodiment of the present invention.

FIG. 6 is a schematic diagram illustrating the outer appearance of an automatic vending machine of urea water, in which a urea water manufacturing device according to an exemplary embodiment of the present invention is equipped.

### DETAILED DESCRIPTION OF THE EMBODIMENTS

The terminology used herein is merely to refer to a particular exemplary embodiment and is not intended to limit the present invention. Singular expressions used herein include plural expressions unless they have definitely opposite meanings. As used in the specification, the meaning of “comprising” specifies a particular characteristic, region, integer, step, action, element, and/or component, and does not exclude the presence or addition of other particular characteristics, regions, integers, steps, actions, elements, components, and/or groups.

Although not defined otherwise, all terms including technical and scientific terms used herein have the same meaning as commonly understood by one of ordinary skill in the art to which the present invention belongs. Terms defined in commonly used dictionaries are additionally interpreted to have a meaning consistent with the related technical literature and the presently disclosed contents, and are not interpreted in an ideal or very formal sense unless defined.

The present invention will be described more fully hereinafter with reference to the accompanying drawings, in which exemplary embodiments of the invention are shown. As those skilled in the art would realize, the described embodiments may be modified in various different ways, all without departing from the spirit or scope of the present invention.

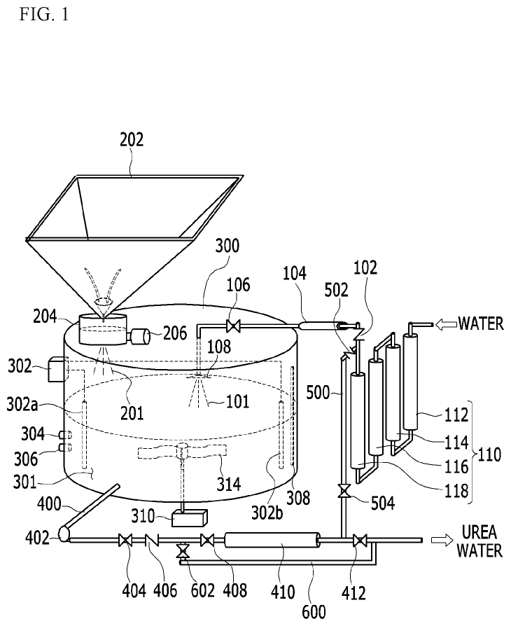

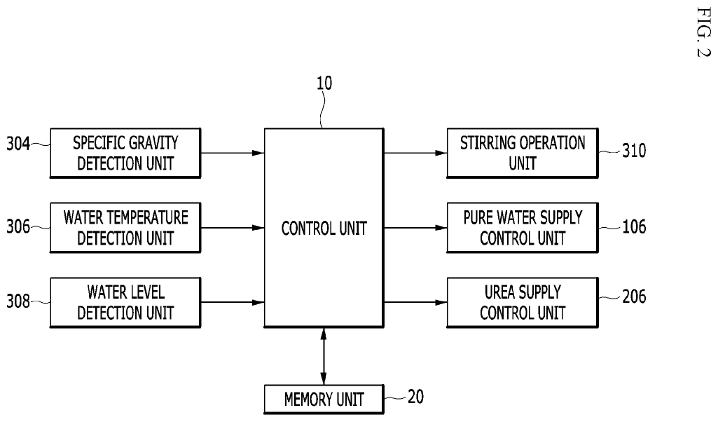

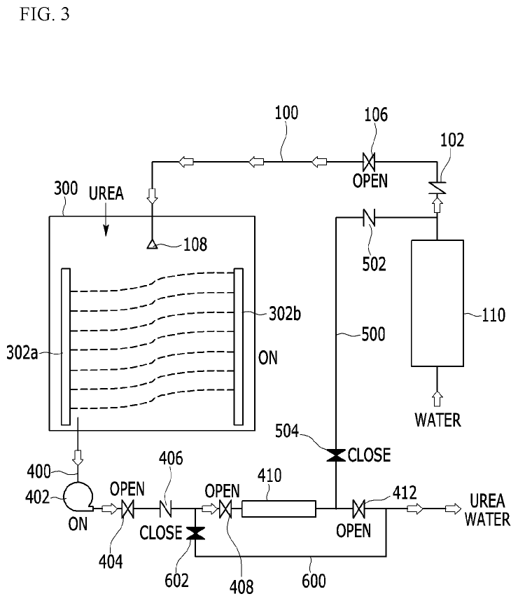

FIG. 1 is a drawing illustrating a urea water manufacturing device according to an exemplary embodiment of the present invention, and FIG. 2 is a block diagram illustrating the control relationship of the urea water manufacturing device according to the present invention. In addition, FIG. 3 is a block diagram illustrating the process of discharging urea produced by the supply of urea and pure water in the process of manufacturing urea according to an exemplary embodiment of the present invention. Referring to FIG. 1 to FIG. 3, the urea water manufacturing device according to the present invention includes a pure water supply unit, a urea supply unit, a stirring unit, a specific gravity detection unit 304, a control unit 10, a urea water discharge unit, and a cleaning unit. The urea water manufacturing device can detect in real time the specific gravity of urea water 301 generated by stirring pure water 101 and urea 201, which are supplied to the stirring tank 300, in a vibrating atmosphere. In addition, high-purity urea water can be manufactured by controlling the specific gravity of urea water 301 to a range of predetermined discharge specific gravity and finally discharging the same. The urea water manufacturing device can be down-sized and its movement can easily be implemented, and thus, high-purity urea water can be manufactured in real time without restriction on the place of installation. Meanwhile, the urea water manufacturing device may directly supply the particulated urea and temperature-controlled pure water to the stirring tank 300. Therefore, the urea water manufacturing device can be implemented in a smaller size.

The pure water supply unit may include a pure water filtration unit 110, a pure water supply control unit 106, an injection unit, a temperature control unit 104, and a pure water supply line 100 which is connected with the stirring tank 300. The pure water supply unit may supply pure water from the outside through the pure water supply line 100 into the stirring tank 300. As the pure water supply unit, any device which can supply pure water to the stirring tank 300 through the pure water supply line 100 can be used. In particular, pure water may include ultrapure water with electrical resistance of 17 MΩ or more. In addition, it is desirable to supply pure water directly through the pure water supply unit, but it is also possible to substitute dissolved water (e.g., distilled water, tap water, ground water, industrial water, etc.) with pure water to be used. As described above, general tap water may be purified through the pure water supply line 100 and supplied as pure water. However, if the tap water is used, the temperature of the supplied water may vary depending on the season. Accordingly, even if the tap water is purified through the pure filtration unit 110 and supplied into the stirring tank 300, the temperature of the pure water may vary. The temperature control unit 104 is provided between the pure water filtration unit 110 and the pure water supply control unit 106 in the pure water supply line 100, and thus, can control the temperature of the pure water to a predetermined supply temperature. Since the temperature and the specific gravity of pure water are correlated, it is advantageous to maintain the temperature of the pure water being supplied in the same manner regardless of the season for manufacture of high purity urea with a constant urea content. For this reason, the pure water supply line 100 may include the temperature control unit 104, which includes a temperature sensor and a Peltier element, to control the temperature of the pure water supplied to the stirring tank 300 to be supplied within a set temperature range. In particular, the temperature of the pure water may be set to 17° C.±1° C. considering the dissolution time of urea without dissolving of impurities regardless of the temperature of tap water according to the season. The temperature control unit 104 provided in the pure water supply line 100 may include a rapid heating device and a cooling device using a Peltier device. It is possible to minimize the change in urea content according to the specific gravity by maintaining the temperature of the pure water being supplied into the stirring tank 300 at 17° C. regardless of the season, using the temperature control unit 104. The effect of low temperature stirring can be enhanced by maintaining the temperature of the pure water being supplied at 17° C. by installing the temperature control unit 104 using a Peltier device in the pure water supply line 100. As such, it is possible to create advantageous conditions for purifying impurities such as biuret and reduce the manufacturing time.

The pure water filtration unit 110 is provided on the inlet side of the pure water supply line 100, and functions to purify the water being supplied from the outside into pure water by filtration. The pure water filtration unit 110 may include an ACfilter 112, an MFfilter 114, an ROfilter 116, and an ion exchanger 118.

The pure water supply control unit 106 may include a pure water control valve, which controls the supply amount of the pure water supplied through the pure water filtration unit 110. In addition, the pure water supply line 100 may further include a supply check valve 102. Upon necessity, a pressure switch may be further provided in the pure water supply line 100. In such a case, the pressure switch may be provided between the pure water supply control unit 106 and the temperature control unit 104.

The injection unit may include an injection nozzle which is provided on the outlet side of the pure water supply line 100 and that injects pure water into the stirring tank 300.

The urea supply unit includes a urea storage unit 202, a particle forming unit 204, a urea supply control unit 206, and a urea supply line connected with the stirring tank 300 and that supplies urea into the stirring tank 300. The urea supply unit may supply particulated urea through the particle forming unit 204, or may directly supply urea that is particulated in advance into the stirring tank 300. The particulated urea is advantageous for supplying an accurate amount by feedback control, and is effective in reducing the stirring time for being able to make the surface area of a solute large.

The urea storage unit 202 stores urea supplied into the stirring tank 300. The urea storage unit 202 has a shape in which the top portion is wide open and the bottom portion is narrowly open. The urea storage unit 202 may be formed of a structure, which has an inclined shape and the stored element is moved toward a lower direction by gravity. The particle forming unit 204 includes a pulverizing unit, and functions to form the urea stored in the urea storage unit 202 into particles. The urea supply control unit 206 functions to supply an accurate amount of as much as a predetermined amount of the particulated urea through the particle forming unit 204 to the stirring section. The urea supply control unit 206 may include a step motor capable of duty control for the supply of an accurate amount of the particulated urea. The step motor is provided at the outlet of the urea supply unit, and can control so that the supply of an accurate amount of particulated urea can be maintained by the control unit 10. As described above, the supply amount of pure water and the supply amount of urea may be controlled to be supplied to the stirring tank 300 at the correct ratio so as to maintain the desired mixing ratio of urea water.

The stirring unit may include a stirrer, a stirring operation unit 310, and a stirring vibration unit 302. In the stirring unit, the pure water 101 and the urea 201 may be stirred in a predetermined stirring vibration atmosphere. In the stirrer, one side of the rotating shaft is coupled to the stirring operation unit 310, and the delivered torque rotates the other end thereby rotating the pure water 101 and the urea 201. The other side of the rotating shaft of rotation may include a plurality of wings 314. The stirring operation unit 310 may include an electric motor which is provided on the outside of the stirring tank 300 and generates rotational power of the stirrer. The operation of the stirrer employs the torque of the stirring operation unit 310, and the rotation speed of the stirring operation unit 310 can be controlled according to the viscosity of the urea water 301 generated in the stirring tank 300. The stirring vibration unit 302 may include ultrasonic wave generators 302a and 302b that generate vibration waves. The stirring vibration unit 302 may form a stirring vibration atmosphere for stirring the pure water 101 and the urea 201 within the stirring tank 300. The ultrasonic wave generators 302a and 302b can reduce the manufacturing time by low temperature stirring. The time for manufacturing the urea water 301 can be reduced by the ultrasonic wave generators 302a and 302b and the supply of the particulated urea in the stirring tank 300, without a separate device for generating a vortex such as a compressor to enhance the effect of low temperature stirring. The ultrasonic wave generators 302a and 302b installed inside the stirring tank 300 can increase the activity of urea molecules by simultaneously generating a high frequency and vibration. In addition, the time for manufacturing the urea water 301 can be reduced by improving the mixing and dissolution rate of the pure water 101 and the urea 201. The ultrasonic wave generators 302a and 302b, during the stirring function, generate vibration waves with a relative low frequency band in the high frequency long wave regions (i.e. 30-300 KHz) so that the urea 201 can be well dissolved in the pure water 101 by bubbles caused by cavitation. In another way, the ultrasonic wave generators 302a and 302b, during the self-cleaning function, generate vibration waves with a relative high frequency band in the high frequency long wave regions so that many bubbles with high penetration intensity and high density are generated by cavitation and used to clean the stirring tank 300. The ultrasonic wave generators 302a and 302b can reduce the stirring time and improve the cleaning efficiency of the stirring tank 300 and the urea water discharge line 400. The stirring unit may further include a water level detection unit 308 and a water temperature detection unit 306. The water level detection unit 308 may include a water level detection sensor which detects the water level of the pure water stored in the stirring tank 300. The water level detection unit 308 can detect, in real time, the water level of pure water 101 or urea water 301 stored in the stirring tank 300. The control unit 10 can control the supply amount of pure water at a predetermined appropriate level by operating the pure water supply control unit 106 according to the detected water level. In addition, the water temperature detection unit 306 may include a water temperature detection sensor which detects the temperature of the urea water 301 as well as the temperature of the pure water 101 stored in the stirring tank 300. The water temperature detection unit 306 can detect, in real time, the temperature of the urea water 301 generated in the stirring tank 300. The control unit 10 can control the temperature of the urea water 301 generated in the stirring tank 300 to be equal to or lower than the predetermined temperature of 17° C., by controlling the temperature of the pure water 101 supplied through the temperature control unit 104 according to the detected temperature of the urea water 301.

Urea water can be stored for 6 months to 2 years depending on the storage conditions. However, unsealed urea water products cannot be reused after storage because they can cause quality deterioration problems due to evaporation of moisture or introduction of impurities therein. Therefore, it is advantageous to produce and supply urea water as needed whenever it is needed from the aspects of cost and inventory management. In addition, the quality of urea water must meet the quality requirements of KS R ISO 22241-1. For urea water manufacturing devices which cannot check the quality of the urea water being generated in real time, the quality of the urea water can be confirmed only via routine consultation of tests with the Korea Petroleum Quality & Distribution Authority with regard to the failure to meet the standard due to a malfunction of the machine or a manager's mistake (specified value: 31.8% to 33.2% of urea content). In this case, some substandard products may be sold until the test results are obtained. Therefore, it is necessary to employ a system that can check and calibrate the quality of the generated urea water in real time.

Meanwhile, a urea water quality sensor for vehicles (ultrasonic quality piezo sensor) may be used to measure the content of the urea water, but measurement accuracy is at the level of ±1%. That is, conventional urea water quality sensors for vehicles can only determine diluted urea and water, and thus it is necessary to measure the urea content in the urea water with more accuracy. Therefore, to manufacture high-purity urea water, the specific gravity detection unit 304 and the water temperature detection unit 306 may be employed. The water temperature detection unit 306 is provided inside the stirring tank 300 and functions to detect the temperature of pure water stored or urea water being generated, for accurate manufacture of the urea water so that the urea content in the urea water can be maintained in a range of 32.3% to 32.7%. The pure water supply temperature may be set based on 17° C., and may be manufactured using the specific gravity target value of 1.31 sg (corresponding to 32.5% of urea water) when urea water is manufactured using pure water at 17° C. In addition, the change in specific gravity of urea water due to the endothermic reaction during stirring of urea and pure water can also be calibrated through the water temperature detection unit 306.

The specific gravity detection unit 304 detects the specific gravity of urea water generated by mixing pure water and urea stirred within the stirring tank 300, and generates the corresponding specific gravity detection signal. The specific gravity detection unit 304 performs the function of detecting the specific gravity of the urea water stirred within the stirring tank 300 for accurate manufacture of urea water so that the urea content in the urea water can be in a range of 32.3% to 32.7%. After the real-time measurement of the specific gravity of the urea generated in the stirring tank 300 through the specific gravity detection unit 304, the supply amount of urea and pure water can be calibrated. Therefore, it is possible to more easily manufacture urea water products that satisfy the urea content specification of 32.5% when manufacturing urea water. The specific gravity detection unit 304 may include a hydrometer using a refractive index. In particular, the hydrometer may include a digital hydrometer. The specific gravity detection unit 304 can accurately measure the amount of solid urea dissolved in pure water as a solution in the stirred tank 300. The specific gravity detection unit 304 may be provided so that the specific gravity measurement range can be maintained in a range of 1.00 sg to 1.50 sg, with accuracy within +0.003 sg at the minimum 0.001 sg unit.

The control unit 10 refers to a logical part of a program that performs a specific function in a computer, in which calculation, treatment, etc. are performed by the processor of an information processing device, and may be implemented by software, hardware, etc. For example, the information processing device may include a personal computer, a handheld computer, a personal digital assistant (PDA), a mobile phone, a smart device, a tablet, etc. In addition, the control unit 10 may further include a memory unit 20 for storing data related to urea water manufacture. The memory unit 20 is a device for storing information, and may include various kinds of memory, such as a high-speed random access memory, a magnetic disk storage device, a flash memory device, other non-volatile solid-state memory devices, etc. The control unit 10 can perform a series of control actions related to manufacture of urea water. For example, the control unit 10 can analyze the specific gravity detection signal supplied from the specific gravity detection unit 304, compare the detected specific gravity of the urea water with a predetermined discharge range of specific gravity, and generate a corresponding feedback control signal thereof so that the detected specific gravity of the urea water can be included in the predetermined discharge range of specific gravity, thereby controlling the specific gravity of urea water produced within the stirring tank 300. The discharge specific gravity of urea water can be set to a range of 1.305 sg to 1.315 sg. In particular, when the detected specific gravity of the urea water exceeds 1.315 sg, the control unit 10 can perform a series of control actions of supplying a pure water supply feedback control signal to the pure water supply unit and thereby further supplying pure water into the stirring tank 300. In addition, when the detected specific gravity of the urea water is lower than 1.305 sg, the control unit can perform a series of control actions of supplying a urea supply feedback control signal to the urea supply unit and thereby further supplying urea into the stirring tank 300. Urea water with a specific gravity above 1.315 sg has an excessive urea content and may cause an ammonia slip phenomenon, in which the ammonia NH₃ remaining after the removal of nitrogen oxides (NOₓ) from a vehicle after-treatment device is released into the atmosphere. In contrast, when the detected specific gravity of the urea water is lower than 1.305 sg, the urea content in urea water is low, and thus the amount of ammonia generated is not sufficient to reduce nitrogen oxides generated in diesel engines, thereby deteriorating the efficiency of purifying harmful exhaust gases. Therefore, at the time of stirring the pure water and urea supplied into the stirring tank 300, the amount of urea or pure water can be precisely controlled to be increased or decreased so that the specific gravity value of urea water generated in the temperature range of the predetermined pure water can be maintained within the discharge specific gravity range of 1.305 sg to 1.315 sg. The control unit 10 may manufacture the urea water using the time difference table control method of the ultrasonic wave generators 302a and 302b and the pure water supply control unit 106.

The urea water discharge unit includes a urea water discharge line 400 connected with the stirring tank 300 and that can discharge the urea water generated in the stirring tank 300 to the outside of the stirring tank 300 through the urea water discharge line 400. The urea water discharge unit may further include a filtration unit, which is provided in the urea water discharge line 400 and that filters impurities being discharged through the urea water discharge line 400. The urea water discharge line 400 may be provided with a booster pump 402, a discharge check valve 406, a discharge solenoid valve, and a UF filter 410. The discharge solenoid valve may include a first discharge solenoid valve 404 which regulates the flow of the urea water being discharged from the booster pump 402, a second discharge solenoid valve 408 which regulates the flow of the urea water flowing into the UF filter 410, and a third discharge solenoid valve 412 which regulates the flow of the urea water discharged from the UF filter 410. In addition, a bypass line 600 can be provided separately so that any residual urea water and discharged products such as impurities can be bypassed without passing through the UF filter 410, by directly connecting the inlet and outlet sides of the UF filter 410. In this case, the bypass line 600 may be provided with a bypass valve 602 which regulates the flow of the bypassed discharged products. If necessary, a pressure switch may be further provided on the urea water discharge line 400. In this case, the pressure switch may be provided between the booster pump 402 and the first discharge solenoid valve 404.

The cleaning unit may include a cleaning line 500, which is connected with the pure water supply line 100 and the urea water discharge line 400. The cleaning unit can selectively remove the urea water and impurities remaining in the stirring tank 300 or the urea water discharge line 400 using the pure water supplied through the pure water supply line 100. The control unit 10 can control the opening and closing operations of a cleaning check valve 502 and a cleaning control valve 504 provided in the cleaning line 500 to remove urea water residues and impurities remaining in the urea water discharge line 400, after manufacturing urea water through the urea water manufacturing process. The control unit 10 can control the overall self-cleaning action. The cleaning of the stirring tank 300, etc. can easily be performed by applying the self-cleaning function to the urea water manufacturing device as such. In addition, it is also advantageous from the aspect of maintenance in that it can easily remove impurities from the urea water discharge line 400 thereby reducing the causes of failures.

Referring to FIG. 1 to FIG. 3, the method for manufacturing urea water according to an exemplary embodiment of the present invention will be described. The method for manufacturing urea water may include a step of supplying pure water, a step of supplying urea, a step of stirring, a step of detecting specific gravity, a step of controlling specific gravity of urea water, and a step of discharging urea water.

First, pure water can be supplied into the stirring tank 300 through the pure water supply line 100 connected with the stirring tank 300 in the step of pure water supply. The water that can be supplied from the outside can be purified to pure water through the pure water filtration unit 110. In addition, the purified pure water is injected into the stirring tank 300 through an injection nozzle 108 via the supply check valve 102 and the pure water supply control unit 106, which are provided in the pure water supply line 100. In particular, the cleaning control valve 504 in the cleaning line 500 is maintained in a closed state. Then, the urea may be supplied into the stirring tank 300 through the urea supply line connected with the stirring tank 300 in the step of urea supply. The supply of pure water and urea may be performed in reverse order, or may be carried out simultaneously, as necessary. In the step of stirring, the pure water and urea supplied into the stirring tank 300 may be stirred in a predetermined stirring vibration atmosphere. Pure water and urea may be stirred in the stirring vibration atmosphere inside the stirring tank 300 and thereby high-quality urea water can be manufactured. In the step of detecting specific gravity, a specific gravity detection signal may be generated by detecting the specific gravity of urea water generated by mixing pure water and urea that are stirred in the stirring tank 300. The control unit 10 can compare the specific gravity of the urea water detected in the step of specific gravity detection with a predetermined discharge range of specific gravity, and generate a corresponding feedback control signal thereof so that the detected specific gravity of the urea water can be included in the predetermined discharge range of specific gravity. In addition, the urea water specific gravity control step of controlling the specific gravity of the urea water generated in the stirring tank 300 can be performed. In particular, the discharge specific gravity of urea water may be set in a range of 1.305 sg to 1.315 sg. In the step of controlling specific gravity of urea water, when the detected specific gravity of the urea water exceeds 1.315 sg, the control unit 10 can generate a pure water supply feedback control signal and thereby further supply pure water into the stirring tank through the pure water supply line 100. In addition, when the detected specific gravity of the urea water is lower than 1.305 sg, the control unit 10 can supply a urea supply feedback control signal and thereby further supply urea into the stirring tank 300 through the urea supply line.

In the step of discharging urea water, the urea generated within the stirring tank 300 can be discharged to the outside of the stirring tank 300 through the urea water discharge line 400 connected with the stirring tank 300. The step of discharging urea water may further include a step of urea water filtration, in which the discharged urea water is filtered using a filtration unit which is provided in the urea water discharge line 400 and that filters impurities being discharged through the urea water discharge line 400. The urea water discharged through the urea water discharge line 400 may be discharged to the outside through the booster pump 402, the first discharge solenoid valve 404, the discharge check valve 406, the second discharge solenoid valve 408, the UF filter 410, and the third discharge solenoid valve 412. In particular, the bypass valve 602 provided in the bypass line 600 is maintained in a closed state.

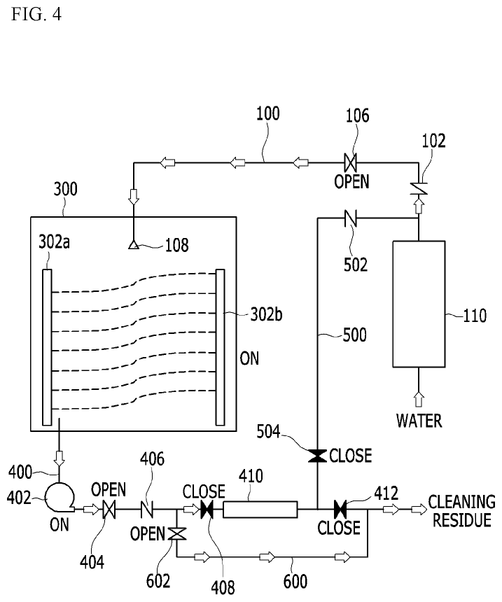

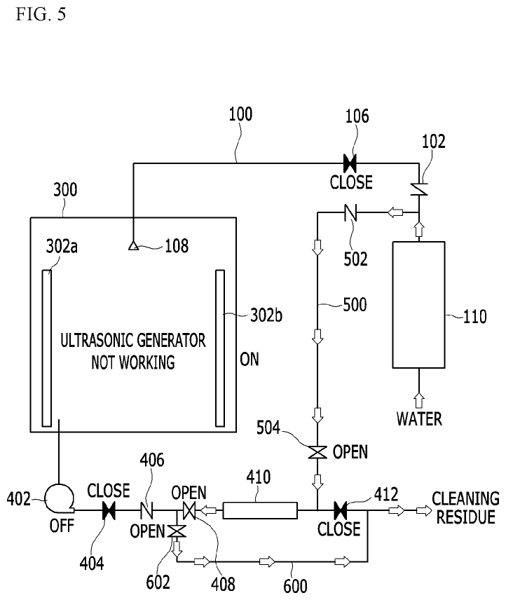

Meanwhile, the control unit 10 can perform, upon necessity, a step of cleaning, in which the urea water remaining in the stirring tank 300 or the urea water discharge line 400 is selectively removed through the cleaning line 500, being connected with the pure water supply line 100 and the urea water discharge line 400, using pure water supplied from the pure water supply line 100. FIG. 4 is a schematic diagram illustrating the process of cleaning a stirring tank using a cleaning unit and an ultrasonic wave generator according to an exemplary embodiment of the present invention, and FIG. 5 is a schematic diagram illustrating the process of cleaning a urea water discharge line using a cleaning unit according to an exemplary embodiment of the present invention.

First, referring to FIG. 4, the cleaning unit may include the pure water supply line 100, the urea water discharge line 400, and the cleaning line 500. The cleaning unit can be operated in conjunction with the operation of the ultrasonic wave generators 302a and 302b and the pure water supply unit for performing the self-cleaning function. The water that can be supplied from the outside is purified to pure water through the pure filtration unit 110, and is injected into the stirring tank 300 through the injection nozzle 108 via the supply check valve 102 and the pure water supply control unit 106, which are provided in the pure water supply line 100. In particular, the cleaning control valve 504 in the cleaning line 500 is maintained in a closed state. The ultrasonic wave generators 302a and 302b, so as to increase the self-cleaning function in an operation state, form a vibrating atmosphere inside the stirring tank 300. The operation of the ultrasonic wave generators 302a and 302b can generate bubbles, thereby increasing the cleaning power. As such, for the self-cleaning function within the stirring tank 300, the cleaning residues such as urea water and impurities remaining inside the stirring tank 300 can be removed by the operation of the ultrasonic wave generators 302a and 302b and the supply of pure water, and discharged through the urea water discharge line 400. The discharged products removed from the stirring tank 300 can be discharged to the outside via the booster pump 402, the first discharge solenoid valve 404, the discharge check valve 406, and the bypass valve 602. In particular, the second discharge solenoid 408 and the third discharge solenoid 412 may be maintained in a closed state to block the inlet of the discharged products into the UF filter 410 thereby preventing the contamination of the UF filter 410.

Referring to FIG. 5, the process of cleaning the urea water discharge line 400 and the UF filter 410 is described. The water that can be supplied from the outside is purified to pure water through the pure filtration unit 110 and is supplied into the UF filter 410 via the cleaning check valve 502 and the cleaning control valve 504, which are provided in the cleaning line 500. In particular, the pure water supply control unit 106 in the pure water supply line 100 is maintained in a closed state. As such, as pure water is supplied to the inside of the UF filter 410, the urea water and impurities remaining in the UF filter 410 can be removed and discharged to the outside of the UFfilter 410. The discharged products removed from the inside of the UF filter 410 can be discharged to the outside via the second discharge solenoid valve 408 and the bypass valve 602. In particular, the first discharge solenoid valve 404 may be maintained in a closed state and block the inlet of the discharged products into the booster pump 402, thereby preventing the contamination of the booster pump 402. In addition, the third discharge solenoid valve 412 may also be maintained in a closed state and block the inlet of the discharged products into the UF filter 410, thereby preventing contamination of the UF filter 410.

As described above, the urea water manufacturing device according to an exemplary embodiment of the present invention can perform cleaning work using the pure water supply line 100 and the urea water discharge line 400, and the pure water being supplied through the cleaning line 500. For example, the urea water and impurities remaining in the stirring tank 300 can be thoroughly removed with pure water, through the time difference table control of the booster pump 402 provided in the urea water discharge line 400, a plurality of discharge solenoid valves 404, 408, and 412, the bypass valve 602 provided in the bypass check valve 406 and the bypass line 600, the injection nozzle 108 provided in the pure water supply line 100, and the ultrasonic wave generators 302a and 302b. The cleaning work using the cleaning unit may be performed so that the steps of preparing the urea water, cleaning the stirring tank 300, and cleaning the urea water discharge line 400 and the UF filter 410 may be converted to the time difference table control. Through the cleaning work, precipitated impurities can be purified using the UF filter 410, which is used in water purifiers in the manufacture of urea water. In addition, at the time of self-cleaning, a plurality of discharge solenoid valves 404, 408, and 412, the discharge check valve 406, the bypass valve 602, etc. may be selectively operated to implement the self-cleaning action. For example, the stirring tank 300 may be cleaned first by the time difference table control during the cleaning work. In addition, the urea water, impurities, etc. remaining in the stirring tank 300 and the urea water discharge line 400 can be removed by cleaning the urea water discharge line 400. In addition, since the inside of the stirring tank 300 can easily be cleaned, any one who does not have general knowledge in the manufacture of urea water can also easily perform the cleaning work. The urea water discharge line 400 used in a particularly down-sized urea water manufacturing device can use a pipe with a diameter of 6 mm to 12.7 mm available at a water purifier level, so failure to clean the device in an appropriate time may result in blockage and failure. As such, the self-cleaning function can prevent the clogging phenomenon occurring in the pipe of the urea water discharge line 400 due to the residual urea water remaining after the manufacture of urea water. That is, to prevent the clogging caused by solidification of the urea water remaining in the stirring tank 300 or the urea water discharge line 400 after the manufacture of urea water, the self-cleaning function can be implemented to automatically remove residual urea by controlling the supply of pure water to a place where residual urea can occur.

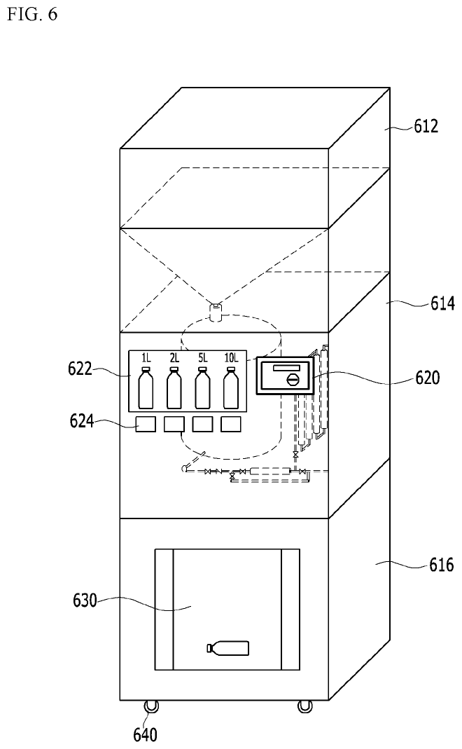

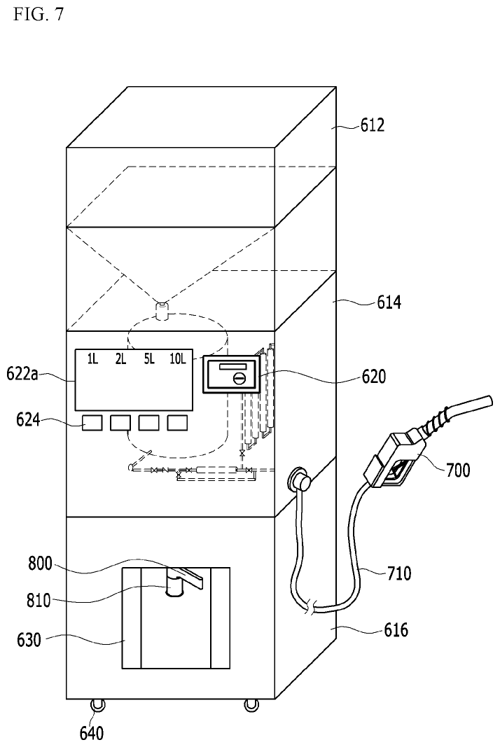

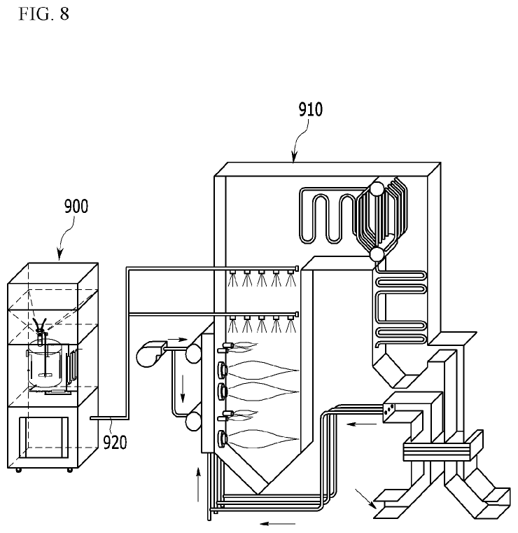

FIG. 6 is a schematic diagram illustrating the outer appearance of an automatic vending machine of urea water, in which a urea water manufacturing device according to an exemplary embodiment of the present invention is equipped. Referring to FIG. 6, the entire size of the urea water manufacturing device may be formed to be as large as a water purifier used at home. Therefore, the urea water manufacturing device may be installed in a narrow space between lubricators at a self-service gas station. The urea water manufacturing device can be down-sized and can manufacture high-purity urea water in real time, and thus the device can have the form of a beverage or coffee vending machine and the urea water generated therefrom in real time can be sold. Since the urea water manufacturing device can be implemented in a form of being embedded in an automatic vending machine, it is possible to easily purchase high-purity urea generated in real time by applying a bill insert or a credit card payment system thereto. For example, the urea water vending machine can be used to sell urea water products, in which urea water generated by a built-in urea water manufacturing device in real time is filled in containers, when the amount of money for the product to be sold is deposited into the device. The urea water vending machine may be provided with a urea storage unit 202, a urea water manufacturing device, and the vend product outlet 630 inside the case. For example, an upper portion 612 may be provided with the urea storage unit 202, and a middle portion 614 may be provided with the urea water manufacturing device. A lower portion 616 may be provided with the product take-out unit 630 so that a purchaser can receive the urea water produced in a designated urea bottle or take the manufactured urea water through the function of automatic packaging with vinyl. The vend product outlet 630 may be provided at one side of the case and formed in a structure that can be opened or closed.

Meanwhile, the front portion of the case may be provided with a cost processing unit 620 that can process the cost with money or a credit card, a display unit 622 for urea water product samples, a selection unit 624 for selecting the urea water products (e.g., push button), etc. In addition, a display unit for displaying information related to urea water manufacture may be provided. In the case, an automatic vending control unit for controlling the overall operation of the urea water vending device based on a predetermined program or data may be provided. The automatic vending control unit may be electrically connected to the control unit 10, and may display a series of manufacturing conditions related to the manufacture of the urea water through the display unit. In addition, the failure details of the urea water manufacturing device may be displayed at the time of failure of the device. In addition, the display unit may be implemented as a touch pad, and may be utilized as an input means for a control function and a self-cleaning function when urea water is manufactured. Meanwhile, the automatic vending control unit may be implemented to be freely installed in various places by attaching wheels 640 thereto, which are moving units at the bottom of the case.

As described above, the urea water manufacturing device according to an exemplary embodiment of the present invention can be implemented at about the size of a water purifier, and thus, it is easy to choose an installation place for the device and it is also easy to move the device to another place if necessary. In addition, since the urea water is produced in real time according to the automatic control operation of the control unit 10, the urea water can be easily and cheaply purchased anywhere in public parking lots, gas stations, and marts. In addition, it is possible to sell urea water products by implementing a urea water automatic vending device. For example, the device can be designed to sell a variety of products from 1 to 10 liters so that consumers can purchase urea water products generated in real time as needed, thereby being able to solve the problems in storage and disposal of urea remaining after use.

FIG. 7 is a schematic diagram illustrating another embodiment of the automatic vending machine of urea water, in which a urea water manufacturing device according to an exemplary embodiment of the present invention is equipped. Referring to FIG. 7, the urea water manufacturing device may include a cost processing unit 620, a urea capacity display unit 622a, a selection unit 624, etc. on a front surface of the case. Additionally, the urea water manufacturing device may include a urea water injection device for injecting urea water into a vehicle. The urea water injection device may include a urea water injection gun 700 and an injection hose 710, which connects the case with the urea water injection gun 700. The urea water injection gun 700 may be implemented by improving the gas gun used in gas stations. Therefore, a user, in a state where the injection amount of the urea water provided in the automatic vending machine is selected and the cost of the urea water is paid, can directly inject urea water into a vehicle using the urea water injection gun 700. The automatic vending machine may include a container injection device for storing urea water in a prepared container. The container injection device may include a discharge pipe 810 through which urea water is discharged and a discharge control valve 800 for controlling discharge of urea water. Therefore, a user can purchase and store urea water required for the prepared container using the container injector when driving a vehicle for a long distance and use the stored urea water when necessary.

FIG. 8 is a schematic diagram illustrating a still another embodiment of the automatic vending machine of urea water, in which a urea water manufacturing device according to an exemplary embodiment of the present invention is equipped. Referring to FIG. 8, an automatic vending machine of urea water 900 may be used by directly connecting it to a selective catalytic reduction (SCR) device or selective non-catalytic reduction (SNCR) device for incinerators, through a supply pipe 920. The automatic vending machine of urea water 900 may be installed in a space from which a large tank for urea water storage used in incinerators is removed. Additionally, since the automatic vending machine of urea water can be installed in a small-sized place such as an apartment-type factory, it is also possible to do business to manufacture and sell urea water directly. As such, the unit cost and logistics cost can be reduced by continuously supplying urea water using the automatic vending machine of urea water.

As described above, the exemplary embodiment of the present invention can reduce the stirring time of pure water and urea without the need for external external device through the ultrasonic wave generators 302a and 302b and urea particularization, and the overall size of the urea water manufacturing device can be down-sized to a size of a domestic water purifier, by feedback calibration of the quality of the generated urea through the specific gravity detection unit 304. Meanwhile, the condition between the specific gravity value of the urea water and the urea content therein can be set so that the specific gravity value of the urea water is manufactured within a predetermined range. More detailed experimental results for manufacturing high-purity urea water in real time are described in the experimental examples below.

### Experimental Example 1

In Experimental Example 1 of the present invention, the specific gravity value of urea water was within the range of 1.305 sg to 1.315 sg discharge specific gravity to test whether the urea water can satisfy the urea content of 31.8% to 33.2%, which is the KS R ISO 22241-1 standard for urea water quality. The relationship between the specific gravity of the urea produced in Experimental Example 1 and the content of urea was measured, and the results are shown in Table 1 below.

TABLE 1

| Category | Experiment 1 | Experiment 2 |
|---|---|---|
| Specific Gravity | 1.307 sg | 1.312 sg |
| Urea Content | 32.2% | 32.4% |

As shown in Table 1, the urea water produced using the urea water manufacturing device according to an exemplary embodiment of the present invention was evaluated by the Research Institute of Petroleum Technology of the Korea Petroleum Quality & Distribution Authority for the test method according to the KS R ISO 22241-2, and the measurement values of the test report are shown. Looking at Table 1, it can be seen that the urea content increases as the specific gravity value increases. Therefore, it was confirmed that setting the specific gravity discharge to a range of 1.305 sg to 1.315 sg during the feedback control enables full satisfaction of the standard of KS R ISO 22241-1.

### Experimental Example 2

In Experimental Example 2 of the present invention, an experiment was performed to confirm the accurate amount of urea supply. To manufacture 32.5% of urea, theoretically, 2.4 kg of urea per 5 kg of pure water is required. The urea water manufacturing device feedback-controls the amount of pure water through the water level detection unit 308, and thus it is possible to use a volume unit of a liter (L) instead of a mass unit of kg. When a mass unit is converted into a volume unit, the density changes according to the temperature, and thus the experiment was performed under the condition of supplying pure water at 17° C. to confirm the subsequent exact amount of urea supply. Since urea undergoes an endothermic reaction upon stirring with pure water, the density of pure water continues to change as it dissolves. Table 2 shows the measurement results of the change in urea content according to the amount of pure water and urea considering the endothermic reaction until the manufacture of urea water is completed.

TABLE 2

| | Experiment | | Experiment | | Experiment | | Experiment | |
|---|---|---|---|---|---|---|---|---|
| Category | 1 | | 2 | | 3 | | 4 | |
| Amount of | 5 | L | 5 | L | 5 | L | 5 | L |
| Pure Water | | | | | | | | |
| Amount of | 2.2 | kg | 2.3 | kg | 2.4 | kg | 2.5 | kg |
| Urea | | | | | | | | |
| Specific | 1.307 | sg | 1.312 | sg | 1.330 | sg | 1.351 | sg |
| Gravity of | | | | | | | | |
| Urea Water | | | | | | | | |

| | | | | |
|---|---|---|---|---|
| Urea Content | 32.2% | 32.4% | 32.9% | 33.5% |

The density of water (1 kg/L at 4° C.) decreases as the temperature increases. Therefore, in the case of 5 L of pure water at 4° C., theoretically, 2.4 kg of urea is required, however, since the actual temperature is higher than 4° C. when stirring urea, the amount of urea required was reduced. Looking at the result values of Experiment 2 in Table 2, it was confirmed that, theoretically, 2.4 kg of pure urea is required to manufacture a 32.4% concentration of urea water, but in fact only 2.3 kg was used. To manufacture 10 L of urea water by applying such ratio, 7.7 L of pure water and 3.54 kg of urea were needed. The experimental values in Table 2 are measured using industrial urea with a nitrogen content of 46.61% which meets the standard of grade 46% of nitrogen N₂. The resulting values may vary depending on the grade of nitrogen content in the urea.

### Experimental Example 3

In Experimental Example 3 of the present invention, an experiment was performed by selecting the conditions to supply the ultrasonic wave generators 302a and 302b and particulated urea to reduce the low temperature dissolving time. The results are shown in Table 3. The ultrasonic wave generators 302a and 302b, as high frequency generators, were able to implement the dissolution rate of urea rapidly and uniformly by penetrating the energy even to a place where the influence by the stirrer is less, using the shock wave of increased pressure and reduced pressure on minute bubbles. The particulated urea being supplied into the stirring tank 300 through the urea supply unit increased the contact area with pure water, and thereby could rapidly implement its dissolution rate. The surface area of the solute was not related to solubility but to dissolution rate. A longer dissolution time can lead to lower production efficiency and may generate salts, as a type of impurities. Under the supply conditions of pure water at 17° C., biuret and triuret are not dissolved in pure water, and thus, high-purity urea water can be produced only by applying a UF filter. In addition, for the manufacture of high-purity urea water, the discharge specific gravity of urea water may be set to a range of 1.305 sg to 1.315 sg under the conditions where pure water at 17° C. is supplied. However, this low temperature dissolving method has a drawback in that it takes a long time to manufacture urea water. The changes in dissolution time were measured where the dissolution time was applied by selectively categorizing the conditions where the ultrasonic wave generators 302a and 302b and particulated urea are supplied, and the results are shown in Table 3 below.

TABLE 3

| | | Ultrasonic | Particulated | Ultrasonic Wave + |
|---|---|---|---|---|
| | Not | Wave | Urea | Particulated Urea |
| Category | Applied | Applied | Supplied | Supplied |
| Dissolution | 21 Min | 17 Min | 16 Min | 12 Min |
| Time | | | | |

As shown in Table 3, by selective application of the ultrasonic wave generators 302a and 302b and particulated urea, the dissolution time measured were revealed, respectively. Looking at the experimental values in Table 3, it was confirmed the application of simultaneous supply of the ultrasonic wave generators 302a and 302b and particulated urea could most effectively reduce the dissolution time.

Through the above-described experimental examples, it is possible to manufacture high-purity urea water in real time by detecting the specific gravity of urea water in real time and performing feedback control related to urea water manufacture using a urea water manufacturing device capable of implementing down-sizing which includes the ultrasonic wave generators 302a and 302b and particulated urea. Since it is possible to down-size the urea water manufacturing device and to manufacture urea water in real time, the device has advantages of easy installation and free movement even in a small space. In addition, it is possible to manufacture urea water whenever necessary regardless of the expiration date, which is advantageous with regard to inventory management. As such, any one who does not have conventional knowledge in urea water manufacture can easily manufacture high-purity urea water and the urea water manufacturing industry can be expanded.

Although the preferred exemplary embodiments of the present invention have been described above, the present invention is not limited thereto, and various modifications can be made within the scope of the claims and the detailed description of the invention and the accompanying drawings, and it is obvious that they also belong to the scope of the present invention.

While this invention has been described in connection with what is presently considered to be practical exemplary embodiments, it is to be understood that the invention is not limited to the disclosed embodiments. On the contrary, it is intended to cover various modifications and equivalent arrangements included within the spirit and scope of the appended claims.

What is claimed is:

1. A urea water manufacturing device, which comprises:
a pure water supply unit, which comprises a pure water supply line connected with a stirring tank and that supplies pure water into the stirring tank;
a urea supply unit, which comprises a urea supply line connected with the stirring tank and that supplies urea into the stirring tank; and
a stirring unit, in which the pure water and the urea supplied into the stirring tank are stirred in a predetermined stirring vibration atmosphere;
wherein the stirring unit includes:
a stirring operation unit configured to generates rotational power of the stirring unit; and
a stirring vibration unit configured to form the stirring vibration atmosphere for stirring the pure water and the urea within the stirring unit,
the stirring vibration unit includes ultrasonic wave generators configured to generate vibration waves, and
wherein a first vibration wave frequency generated by the ultrasonic wave generators during manufacturing the urea water is lower than a second vibration wave frequency generated by the ultrasonic wave generators during self-cleaning.

2. The urea water manufacturing device od claim 1, further comprises:
a specific gravity detection unit, which detects specific gravity of urea water produced by mixing of pure water and urea being stirred inside the stirring tank and generates a corresponding specific gravity detection signal thereof;
a control unit, which analyzes the specific gravity detection signal that is generated in and supplied from the specific gravity detection unit, compares the detected specific gravity of the urea water with a predetermined discharge range of specific gravity, and generates a corresponding feedback control signal thereof so that the detected specific gravity of the urea water is included in the predetermined discharge range of specific gravity, thereby controlling the specific gravity of urea water produced inside the stirring tank; and
a urea water discharge unit, which comprises a urea water discharge line connected with the stirring tank and that discharges the urea water produced inside the stirring tank to the outside of the stirring tank through the urea water discharge line.

3. The urea water manufacturing device of claim 2, wherein
the specific gravity of urea water discharge is in a range of 1.305 sg to 1.315 sg.

4. The urea water manufacturing device of claim 3, wherein,
when the detected specific gravity of the urea water exceeds 1.315 sg, the control unit supplies a pure water supply feedback control signal to the pure water supply unit and thereby further supplies pure water into the stirring tank.

5. The urea water manufacturing device of claim 3, wherein,
when the detected specific gravity of the urea water is lower than 1.305 sg, the control unit supplies a urea supply feedback control signal to the urea supply unit and thereby further supplies urea into the stirring tank.

6. The urea water manufacturing device of claim 2, wherein
the stirring unit comprises:
a stirrer, which rotates as an external force is transmitted thereto and thereby stirs pure water and urea; and
a stirring operation unit, which generates rotational power of the stirrer.

7. The urea water manufacturing device of claim 6, wherein
the stirring unit further comprises a stirring vibration unit, which comprises an ultrasonic wave generator that generates vibration waves, and forms a stirring vibration atmosphere for stirring of the pure water and the urea inside the stirring tank.

8. The urea water manufacturing device of claim 2, wherein
the pure water supply unit comprises:
a pure water filtration unit, which is provided on the inlet side of the pure water supply line and filters the water supplied from the outside into pure water;
a pure water supply control unit, which is provided in the pure water supply line to control the supply amount of pure water filtered through the pure water filtration unit;
an injection unit, which is provided at the outlet side of the pure water supply line and injects pure water; and
a temperature control unit, which is provided between the pure water filtration unit and the pure water supply control unit in the pure water supply line and controls the temperature of pure water to a predetermined supply temperature.

9. The urea water manufacturing device of claim 2, wherein
the urea supply unit comprises:
a urea storage unit, which stores urea supplied to the stirring tank;
a particle forming unit, which forms the urea stored in the urea storage unit in particles; and
a urea supply control unit, which supplies an accurate amount of the particulated urea into the stirring unit.

10. The urea water manufacturing device of claim 2, wherein
the device further comprises a filtration unit, which is provided in the urea water discharge line and filters impurities being discharged through the urea water discharge line.

11. The urea water manufacturing device of claim 2, wherein
the device further comprises a cleaning unit, which comprises a cleaning line connected with the pure water supply line and the urea water discharge line, and that selectively removes the urea water that remains in the stirring tank or the urea water discharge line using the pure water supplied from the pure water supply line.
